(19) United States
(12) Patent Application Publication
(10) Pub. No.: US 2008/0097161 A1
(43) Pub. Date: Apr. 24, 2008

(54) Semi-Flexible Electronic Laryngoscope Bendable Upwardly and Downwardly

(75) Inventors: Soo Geun Wang, Dongnae-gu Busan (KR); Gye Rok Jeon, Saha-gu Busan (KR); Hoon Ro, Nam-gu Busan (KR); Jeong Hwan Ok, Gyeonggi-do (KR)

Correspondence Address: THE WEBB LAW FIRM, P.C., 700 KOPPERS BUILDING, 436 SEVENTH AVENUE, PITTSBURGH, PA, 15219, US

(73) Assignee: Soo Geun Wang, Busan (KR)

(21) Appl. No.: 11/814,326

(22) PCT Filed: Mar. 16, 2005
(86) PCT No.: PCT/KR05/00762

§ 371 Date: Jul. 19, 2007

(30) Foreign Application Priority Data
Jan. 27, 2005 (KR) 10-2005-0007478

(51) Int. Cl. A61B 1/267

(52) U.S. Cl. 600/188

(57) ABSTRACT

Disclosed herein is a semi-flexible electronic laryngoscope comprising in one body a photographing part which can observe the larynx inside the oral cavity with a self-lamination, a flexure part which bends the photographing part upwardly and downwardly by a rotation member that is connected to the photographing part and located at the handle, a stiff part which leads the flexure part to the oral cavity and the larynx and maintains stably, and a handle which accepts and supports the end point of the stiff part, which can observe the larynx and the hy-popharynx when bent downwardly and the nasopharynx when bent upwardly since it can be bent upwardly and downwardly, and can diagnose the oral cavity and the oropharynx since it can observe the forward area when inserted into an oral cavity.

### TECHNICAL FIELD

The present invention relates to an improved semi-flexible electronic laryngoscope bendable upwardly and downwardly. More specifically, the invention relates to such a semi-flexible electronic laryngoscope bendable upwardly and downwardly, by which affected parts of a patient such as the larynx and the nasopharynx are observed correctly, while bending a photographing part upwardly and downwardly by the operation of a flexure part, and in which a photographing part and a flexure part is mounted on the front edge of a solid stiff part so that stable observation is possible when diagnosing the larynx and the nasopharynx; the impact is absorbed, which may be transferred to the throat by the flexure part when a patient moves abruptly; the probability of misdiagnosis is reduced; the time for diagnosing and treating is shortened; and affected parts are easily photographed through a CCD or a CMOS image sensor, and since structural strength is great, the life span is long and the production costs are relatively low.

### BACKGROUND ART

As for throat diseases, a medical specialist examines and treats an affected part through an endoscope when a patient feels bad with throat and visits a special clinic by oneself. The inside of the endoscope that is moved to the affected part of a patient is illuminated by a light source, and the affected part illuminated by the light source are photographed by a camera and displayed on a TV or a PC monitor, making it possible to observe and treat the affected part.

However, a conventional endoscope, configured as above, illuminates inside the human body by a specially equipped light source through an optical fiber, and observes an affected part using expensive optical systems such as an optical fiber bundle or a rod lens, making it hard to be spread easily. The light source illuminating through an endoscope is expensive and is serious in electric power loss due to the continuous supply of much electric power, and a camera system photographing the affected part also is costly and needs to be purchased additionally, thereby imposing an economic burden.

In addition, all kinds of conventional endoscopes are either a bend-type which is bent as a whole or a stiff-type which is not bent at all. In case of the former, when observing the oral cavity, the nasal cavity, the nasopharynx, and the larynx, it is hard to secure a fixed visual field toward the part to be observed, and is inconvenient to use more or less, requiring much time for diagnosing and treating. In case of the latter, it is sometimes difficult to access an affected part at a deep position, and a stiff tube may impact and hurt the pharynx when a patient moves abruptly, raising a safety problem.

According to Korean Patent Laid-Open Publication 2004-52868 previously filed by the present inventor to solve such problems, a semi-flexible electronic laryngoscope capable of observing the larynx comprises in one body a photographing part which can observe the larynx, an operation part which is connected to the photographing part and operates the photographing part upwardly and downwardly, a flexible tube which accepts the operation part so as the photographing part to be protruded and moves upwardly and downwardly according to the action of the operation part, and a handle which accepts and supports the end point of the flexible tube.

However, a conventional semi-flexible electronic laryngoscope bendable upwardly and downwardly is configured so as to be bent upwardly and downwardly with respect to a guide rib, by aligning guide holes of the guide rib of the adjacent operation member with guide holes of the guide rib formed at both sides of the front end of multiple operation members inside the flexible tube and axial-coupling them with a fixing member. However, since multiple ring-type operation members, which form a guide rib having guide holes formed at the upper and the lower end respectively, are used and multiple parts are fastened by the fixing member, the production cost is relatively high, and the axial-coupled fixing member of the guide rib is broken away occasionally during its use, so that the case in which the operation of the flexible tube is insufficient is found and it exhibits weakness in strength.

### DISCLOSURE OF INVENTION

### Technical Solution

The present invention has been made in order to solve the above problems occurring in the art, and it is an object of the invention to provide a semi-flexible electronic laryngoscope bendable upwardly and downwardly, which observes an affected part of a patient correctly, such as the larynx and the nasopharynx while bending a photographing part more than 130 degrees upwardly and downwardly by the operation of a flexure part led to the location for photographing by a stiff part, thereby reducing dominantly the probability of misdiagnosis, easily photographs an affected part through a CCD or a CMOS image sensor with the illumination of an LED, and is integrally formed into an elastic body without using a hinge that becomes a relative motion for connecting the flexure part, thereby extending lifespan and relatively reducing production costs due to great structural strength.

In order to accomplish the above object, according to one aspect of the present invention, there is provided a semi-flexible electronic laryngoscope bendable upwardly and downwardly comprising integrally a photographing part which can observe the larynx, a flexure part which is connected to the photographing part and operates the photographing part upwardly and downwardly, a stiff part which leads the flexure part to the oral cavity and the larynx, and a handle which accepts and supports the end point of the stiff part, wherein the laryngoscope can be bent upward-and-downwardly so that the larynx and the hypopharynx can be observed when bent downwardly and the nasopharynx can be observed when bent upwardly, and the laryngoscope can observe the forward area when inserted into an oral cavity, thereby diagnosing the oral cavity and the oropharynx, wherein the handle of the flexure part includes: a ring-type operation member stacked at least more than one in order to allow the body, which has a hole inside, to move upwardly and downwardly; a pair of spring- or bar-type elastic members which pass through both sides of the stacked body of the operation member, and support to be bent to one side and the other side; a pair of connecting members which connect the operation member in opposite direction in order to be bent in the same direction; and a rotation member which bends a pair of the connecting members upwardly and downwardly.

### BRIEF DESCRIPTION OF THE DRAWINGS

Further objects and advantages of the invention can be more fully understood from the following detailed description taken in conjunction with the accompanying drawings, in which:

FIG. 1 is a perspective view of a semi-flexible digital electronic laryngoscope according to the invention;

FIG. 2 is a separated perspective view of a semi-flexible digital electronic laryngoscope according to the invention;

FIG. 3 is a separated perspective view of a semi-flexible digital electronic laryngoscope showing a photographing part according to the invention;

FIG. 4 is a cross-sectional elevation of a semi-flexible digital electronic laryngoscope according to the invention;

FIG. 5 is a cross-sectional plane of a semi-flexible digital electronic laryngoscope according to the invention;

FIG. 6 is a cross-sectional elevation of a semi-flexible digital electronic laryngoscope showing the state of an end point bent upward according to the invention; and

FIG. 7 is a cross-sectional elevation of a semi-flexible digital electronic laryngoscope showing the state of an end point bent downward according to the invention.

### BEST MODE FOR CARRYING OUT THE INVENTION

The preferred embodiments of the present invention will be hereafter described in detail with reference to the accompanying drawings.

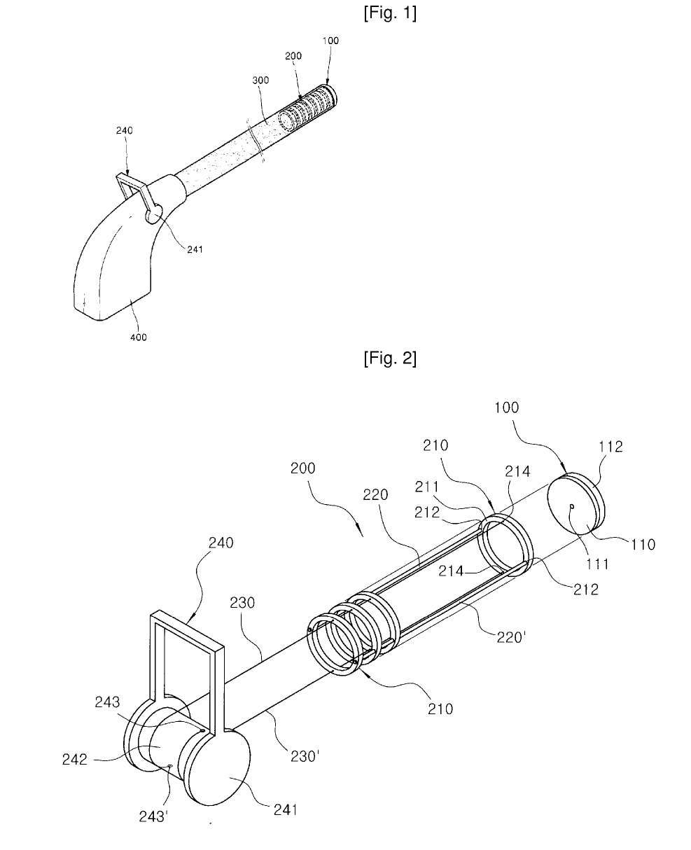

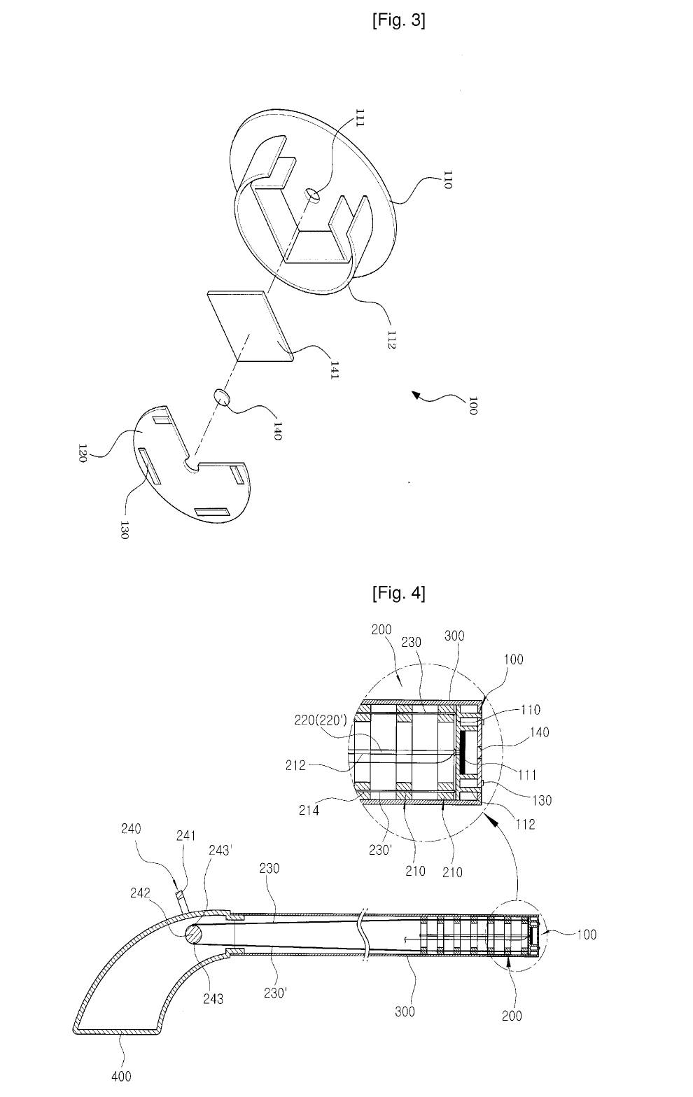

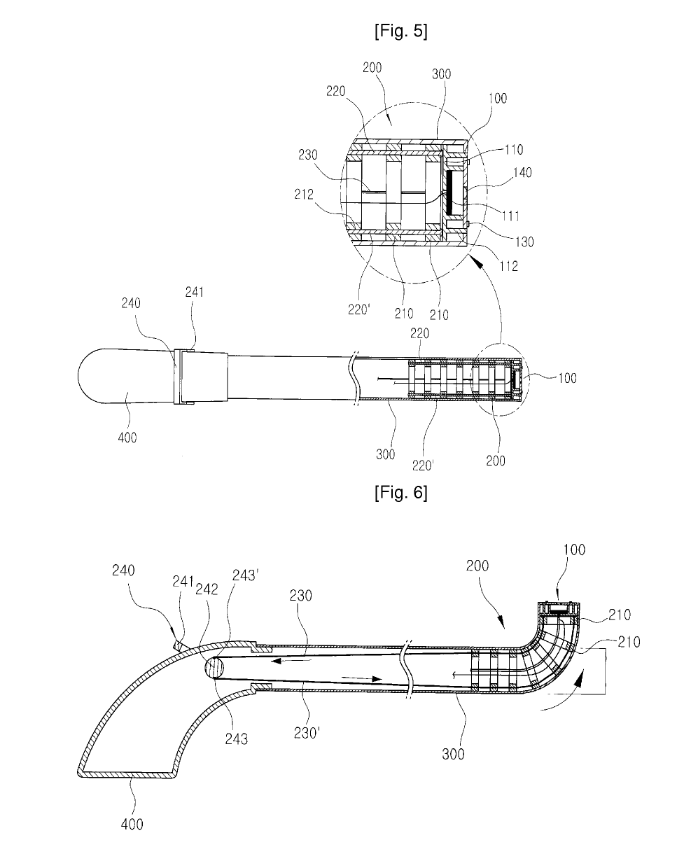

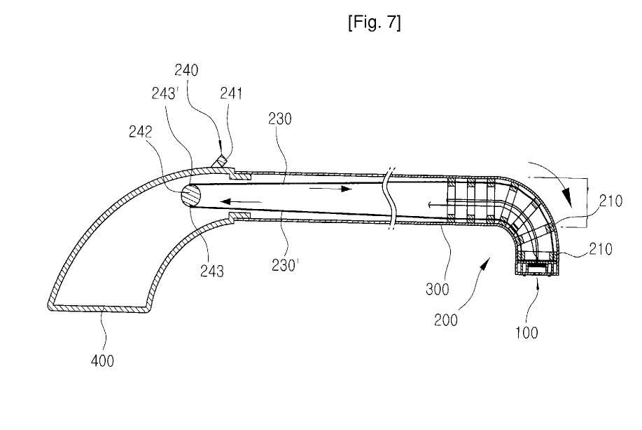

FIG. 1 is a perspective view of a semi-flexible digital electronic laryngoscope according to the invention. FIG. 2 is a separated perspective view of a semi-flexible digital electronic laryngoscope according to the invention. FIG. 3 is a separated perspective view of a semi-flexible digital electronic laryngoscope showing a photographing part according to the invention. FIG. 4 is a cross-sectional elevation of a semi-flexible digital electronic laryngoscope according to the invention. FIG. 5 is a cross-sectional plane of a semi-flexible digital electronic laryngoscope according to the invention. FIG. 6 is a cross-sectional elevation of a semi-flexible digital electronic laryngoscope showing the state of an end point bent upward according to the invention. FIG. 7 is a cross-sectional elevation of a semi-flexible digital electronic laryngoscope showing the state of an end point bent downward according to the invention.

The improved semi-flexible electronic laryngoscope bendable upwardly and downwardly of the invention comprises a photographing part 100 which can observe the oral cavity, the nasopharynx, as well as the larynx, a flexure part 200 which is connected to the photographing part 100 and operates the photographing part 100 upwardly and downwardly, a stiff part 300 of hard material which accepts the photographing part 100 and the flexure part 200 and thus is placed stably while moving upwardly and downwardly according to the operation of a rotation member 240, and a handle 400 which accepts and supports the end point of the stiff part 300.

The flexure part 200 is formed of a ring-type operation member 210 which is stacked at least more than one to allow the soft body 211, which has a hole formed therein, to be bent upwardly and downwardly, a pair of spring- or bar-type elastic members 200 220 which pass through both sides of the body 211 of the stacked operation member 210 and support the body to be bent to one side and the other side, a pair of connecting members 230 and 230 which connects the operation member 210 in opposite direction to be bent upwardly and downwardly, and a rotation member 240 which bends a pair of the connecting members 230 and 230 upwardly and downwardly, which is mounted on the handle 400.

The operation member 210 includes an accepting hole 214 which passes through oppositely the upper and the lower portion of the cylindrical body 211 having a hole inside and then accepts a pair of connecting member 230 and 230, and a fitting hole 212 which passes through oppositely the left and the right side of the body 211 and then accepts a pair of spring- or bar-type elastic member 200 and 200.

The photographing part 100 is formed of a housing 110 which has a hole through the center and a fixed rib 112 protruded on the surface of the circumference, a blocking plate 120 which rests on the fixed rib 112 in order to block the inside and the outside of the housing 110, an illuminating member 130 which is mounted on the blocking plate 120 in a radial shape and illuminates selectively by the operation of a switch, and a CCD or a CMOS image sensor 141 which is mounted at the center of the blocking plate 120 and photographs an affected part illuminated by the illuminating member 130.

Specifically, the illuminating member 130 uses an LED (Light Emitting Diode).

The rotation member 240 mounts the end points of a pair of the connecting members 230 and 230 on the fixing holes 243 and 243 formed in the upper and the lower portion of the axis of rotation 242 respectively, and is formed of a rotating lever 241 which operates the operation member 210 upwardly and downwardly.

The process of assembly of the invention, configured as above descriptions, is explained hereafter.

First, a lens 140 is placed at the center of a blocking plate 120 and then a CCD or a CMOS image sensor 141 is mounted. An LED, an illuminating member 130, is mounted in a radial shape with the lens 140 in the center, and an electric wire is connected to the CMOS image sensor 141 and the illuminating member 130 from the rear portion of the blocking plate 120. In the state of passing through an accepting hole 111 of the housing 110, after passing through the side of the CCD or the CMOS image sensor 141, the electric wire rests on a fixing rib 112. Like this, the photographing part 100 is assembled.

In addition, with the insertion holes 212 aligned on a straight line, which are on the left and the right side of the multiple operation members 210, a pair of the elastic members 220 and 220 are combined with the insertion holes 212. At this time, the multiple operation members 210 are combined so as to be bent upwardly and downwardly with the elastic members 220 and 220 in the center.

In such a way as described above, with the multiple operation members 210 combined with a pair of the elastic members 200 and 200, a pair of the connecting members 230 and 230 pass through each accepting hole 214 of the upper and the lower portion of the multiple operation members 210, and the end points of the connecting members 230 and 230 are coupled with the fixing holes 243 and 243 of the rotation member 240 respectively, so the flexure part 200 is assembled.

Like this, when the assembly of the flexure part 200 has been completed, the wire of the photographing part 100 is moved to the inside of the body 211 of the operation member 210, and the one side of the housing 110 of the photographing part 100 is coupled with the end point of the operation member 210.

In this way, when the photographing part 100 is coupled with the flexure part 200, the flexure part 200 and the photographing part 100 are moved to the stiff part 300 in order that the photographing part 100 is protruded to the end point of the stiff part 300.

In addition, after mounting the rotation member 240, which is combined with the connecting members 230 and 230, on the handle 400 so as to be bendable, the stiff part 300 is combined and fixed inside the handle 400.

Hereafter, the state of using the invention is explained in detail.

First, a doctor holds the handle 400, turns on the illuminating member 130, moves the photographing part 100 to the oral cavity of a patient, then photographs the affected part illuminated by the illuminating member 130 using the CCD or the CMOS image sensor 141, and the photographed image is displayed on a monitor and affected part are observed.

That is, bending the photographing part 100, which photographs an affected part of a patient, upwardly and downwardly by the rotation of the rotation member 240, a doctor observes and operates with ease.

In addition, it goes without saying that the improved semi-flexible electronic laryngoscope bendable upwardly and downwardly of the invention can be utilized widely at a urologic, a dental, an obstetric and gynecologic, an ophthalmic, and a dermatologic clinic.

As explained above, the present invention reduces the probability of misdiagnosis dominantly by forming a flexure part at the front edge of a stiff part and observing an affected part of a patient correctly such as the oral cavity, the oropharynx, the larynx, the hypopharynx, and the nasopharynx while bending a photographing part more than 130 degrees upwardly and downwardly by the operation of the flexure part combined with a elastic member which does not have a hinge part, photographs the affected part easily through a CCD or a CMOS image sensor, diagnoses and treats it while watching the photographed image through a monitor, and is long-lived and relatively low in production costs due to great structural strength.

### INDUSTRIAL APPLICABILITY

As described above, the present invention reduces the probability of misdiagnosis dominantly by observing an affected part of a patient correctly such as the oral cavity, the oropharynx, the larynx, the hypopharynx, and the nasopharynx, bending a photographing part more than 130 degrees upwardly and downwardly, photographs the affected part easily through a CCD or a CMOS image sensor, diagnoses and treats watching the photographed image through a monitor, and is long-lived and relatively low in production costs due to great structural strength. Therefore, an improved semi-flexible electronic laryngoscope bendable upwardly and downwardly of the invention can be utilized widely at a urologic, a dental, an obstetric and gynecologic, an ophthalmic, and a dermatologic clinic.

While the present invention has been described with references to the particular illustrative embodiments, it is not to be restricted by the embodiments but only by the appended claims. It is to be appreciated that those skilled in the art can change or modify the embodiments without departing from the scope and spirit of the present invention.

1. A semi-flexible electronic laryngoscope bendable upwardly and downwardly, which integrally comprises: a photographing part 100 which can observe the larynx, a flexure part 200 which is connected to the photographing part 100 and operates the photographing part 100 upwardly and downwardly, a stiff part 300 which leads the flexure part to the oral cavity and the larynx, and a handle 400 which accepts and supports the end point of the stiff part, wherein the laryngoscope can be bent upwardly and downwardly so that the larynx and the hypopharynx can be observed when bent downwardly and the nasopharynx can be observed when bent upwardly, and the laryngoscope can observe the forward area when inserted into an oral cavity, thereby diagnosing the oral cavity and the oral pharynx,
wherein the flexure part 200 includes:
a ring-type operation member 210 stacked at least more than one in order to allow a body 211, which has a hole formed therein, to move upwardly and downwardly;
a pair of spring- or bar-type elastic members 220 and 220′ which pass through both sides of the body 211 of the stacked operation member 210, and support the body 211 to be bent to one side and the other side;
a pair of connecting members 230 and 230′ which connect the operation member 210 in opposite direction in order to be bent in the same direction; and
a rotation member 240 which bends a pair of the connecting members 230 and 230′ upwardly and downwardly, the rotation member being mounted on the handle 400.

2. The laryngoscope according to claim 1, wherein the operation member 210 includes:
an accepting hole 214 which passes through the upper and the lower portion of the cylindrical body 211 oppositely, which has a hole inside, and accepts a pair of connecting members 230 and 230′; and
a fitting hole 212 which passes through the left and the right side of the body 211 oppositely, and accepts the pair of spring- or bar-type elastic members 200 and 200′.
